(19) United States
(12) Patent Application Publication
(10) Pub. No.: US 2003/0104038 A1
(43) Pub. Date: Jun. 5, 2003

(54) Cough treatment

(75) Inventors: John David Hull, Bracknell Berkshire (GB); Paul John Rennie, Godalming Surrey (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/336,909

(22) Filed: Jan. 6, 2003

Related U.S. Application Data
(63) Continuation of application No. PCT/US01/20495, filed on Jun. 27, 2001.

(30) Foreign Application Priority Data
Jul. 7, 2000 (GB) 00/16693.4

(51) Int. Cl. A61K 9/70

(52) U.S. Cl. 424/443

(57) ABSTRACT

The present invention relates to methods and devices for treating cough, in particular in which a person in need of cough treatment is provided with a buccally adherent patch and is instructed to stimulate the patch when feeling a need to cough. The invention works through triggering natural physiological control mechanisms in human cerebral cortex to control the cough reflex.

### CROSS REFERENCE RELATED TO APPLICATIONS

This is a continuation of International Application PCT/US01/20495 with an international filing date of Jun. 27, 2001.

### FIELD OF THE INVENTION

The present invention relates to methods and devices for treating cough, in particular in which a person in need of cough treatment is provided with a buccally adherent patch and is instructed to stimulate the patch when feeling a need to cough.

### BACKGROUND OF THE INVENTION

Cough is a protective mechanism to guard the airways from irritants. However, pathological cough can be irritating, stressful and non-beneficial both physiologically and socially. People suffering from coughing commonly take cough syrups, drops or lozenges, for symptomatic relief. In some cases, prescription drugs may be used to control the symptoms. It is commonly accepted that coughing, and the throat irritation associated with it, can be treated by lubrication with sweet and viscous syrups. Thus, for example, cough drops or lozenges often contain sugar or honey. Active ingredients can also be incorporated into the syrups or lozenges. These active ingredients include, for example, antitussives, which can act to suppress the cough reflex, such as dextromethorphan and its hydrobromide salt; expectorants, such as guaifenesin, which make the cough more productive; and other agents such as coolants which may act by relieving the throat irritation, menthol, peppermint oil and the like are well known in this regard, other physiological coolants such as menthane carboxamides are also useful in this regard.

Other delivery vehicles that have been used to deliver cough actives include chewing gums, such as those disclosed in U.S. Pat. No. 5,846,557, and mucoadhesive devices such as those disclosed in PCT publication WO 95/05416.

In spite of the many products and disclosures relating to cough treatment the present inventors believe that insufficient attention has been paid to the importance of the cortical control of cough.

The present inventors have found that physical contact with a buccally adhered device triggers natural physiological control mechanisms in human cerebral cortex to control the cough reflex.

It is therefore an object of the present invention to provide a method and devices for the treatment of cough, more particularly to a method, and devices for use in the method, which activates or enhances the cortical control of cough.

These objectives will become readily apparent from the detailed description which follows.

### SUMMARY OF THE INVENTION

The present invention relates to a method of treating cough comprising:

(a) attaching a device to the interior of the oral cavity of a person in need of cough treatment;

(b) providing instructions to the person to stimulate the device as necessary.

The present invention further relates to the use of a mucoadhesive polymer in the manufacture of a device for the treatment of cough, the treatment comprising:

(a) attaching the device to the interior of the oral cavity of a person in need of cough treatment;

(b) providing instructions to the person to stimulate the device as necessary.

All percentages and ratios herein are by weight unless otherwise specified. Percentages are by weight of the total composition unless otherwise specified.

### DETAILED DESCRIPTION OF THE INVENTION

The essential as well as optional components of the compositions of the present invention are described in the following paragraphs.

Device

The methods of the invention of the invention utilise a device which is attached to the interior of the oral cavity of a person in need of cough treatment. The precise nature of the device, and the material from which it is made, is not critical. Clearly it should be safe for use in the oral cavity. That is it should preferably be free of sharp edges and should not cause obstruction of the airway if swallowed. Further, since it is intended that the person in need of treatment will carry the device in their mouth for a considerable period of time, the device should feel comfortable in the mouth and not, for example, cause ‘gagging’.

In the preferred practise of the invention the device is a mucoadhesive gel patch or disc, made entirely from food grade materials such as gelatin, having a thickness of from 1-2 mm and a diameter of from 1 mm to 1 cm. Alternately, the disc can be a laminated device having, for example a water insoluble layer and a mucoadhesive layer. Discs are preferred because of their lack of corners, but the shape is not especially important. Other possible shapes include squares, strips and hoops. The device can optionally be textured, especially on the face directed to the interior of the oral cavity, so that it feels rough to the tongue, in order to provide greater stimulation when touched.

Suitable mucoadhesive polymers for use in the manufacture of the device include polysaccharides such as for example cellulose-type materials and natural gums, polypeptides, and water-soluble synthetic polymers. Particular examples of such suitable polymers which are GRAS certified include poly(vinyl pyrrolidone) (“PVP”), poly(vinyl alcohol) (“PVA”), hydroxy propyl cellulose (“HPC”), poly(ethylene oxide) (“PEO”), poly(acrylic acid) (“PAA”), polyacrylates such as Carbopol 934 (B. F. Goodrich), starch and starch derivatives, polysaccharides, sodium carboxymethyl cellulose (“Na-CMC”), xanthan gum, karaya gum, and gelatin, among others. Preferred is gelatin. Suitable devices are disclosed in U.S. Pat. No. 5,700,478 which is incorporated herein by reference in its entirety. Other suitable devices are available from 3M as the Cydot® TMD System. WO93/23011 also discloses suitable devices and is incorporated herein by reference in its entirety.

The devices of the invention need not contain any active ingredients since they are particularly intended to trigger natural physiological control mechanisms in the human cerebral cortex to control the cough reflex.

It is recognised however that drug based remedies may further enhance their efficacy in controlling cough and any of the usual cough actives known to the skilled person can also be incorporated into the device. Among useful active agents are antitussives, which can act to suppress the cough reflex, such as dextromethorphan and its hydrobromide salt; and expectorants, such as guaifenesin.

Other suitable components can include plasticisers, such as glycerin, sorbitol, and other edible polyhydric alcohols; flavours and other organoleptic stimulants such as physiological cooling and/or warming agents to make the device more palatable in the mouth or to increase the stimulation when the patch is touched; and antibacterial agents such as triclosan or quaternary ammonium salts which can help to reduce associated infections. The devices are preferably sugar-free.

Methods of treatment and manufacture

The basic method of treatment involves attaching the device to the interior of the oral cavity of a person in need of cough treatment and providing instructions to the person to stimulate the device as necessary, in particular when the person feels a need or an incipient need to cough. The preferred method of stimulating the device is for the person to touch the device with their tongue when they feel the need to cough. In doing so the person's tongue is in turn stimulated providing a physiological feedback.

The device can be attached to the oral cavity by any safe method but it is preferred to attach it by adhesion, through the use of a mucoadhesive polymer as described above.

The device is suitably attached in any position where it can be stimulated by the tongue, and preferably by the tip of the tongue. Suitable locations include on the teeth, on the hard palate the inside of the lips and the inside of the cheek, the inside of the cheek being preferred.

In the preferred method the instructions are included with the device, for example by way of a kit comprising device and instructions, for the person to touch the device with their tongue when they feel the need to cough.

The devices can be manufactured using art-known methods such as the drying down of an aqueous solution of a polymer until a plastic disc or sheet is obtained, or by using any of the methods described in the references above.

The following examples further describes and demonstrate preferred embodiments within the scope of the present invention. The examples are given solely for illustration and are not to be construed as limitations of this invention.

### EXAMPLE

A set of flexible gel discs of about 1 cm in diameter and 1 mm thickness were prepared from a 38% solution of Gelatin Bloom 220 in water. A group of ten people suffering from cough were asked to stick a gel disc to the inner, right-hand side of their cheek and to keep the disc in position without sucking or chewing on the disc. They were then instructed “When you feel a cough coming on, instead of coughing, touch the disc with your tongue”. A lower frequency of coughing was recorded within the group than a control group of cough sufferers who were instructed “You have been allocated not to take anything for your cough”.

What is claimed is:

1. A method of treating cough comprising:
a) attaching a device to the interior of the oral cavity of a person in need of cough treatment;
b) providing instructions to the person to stimulate the device as necessary.

2. The method according to claim 1 wherein the device is a food-grade, mucoadhesive patch.

3. The method according to claim 2 wherein the mucoadhesive patch comprises a mucoadhesive polymer.

4. The method according to claim 3 wherein the mucoadhesive polymer is gelatin.

5. The method according to claim 1 further comprising instructing the person to touch the device with their tongue when they feel the need to cough.

6. A method of using a mucoadhesive polymer in the manufacture of a device for the treatment of cough, the method comprising:
a) attaching the device to the interior of the oral cavity of a person in need of cough treatment;
b) providing instructions to the person to stimulate the device as necessary.

7. The method according to claim 6 wherein the mucoadhesive polymer is gelatin.

8. The method claim 7 wherein the method further comprises instructing the person to touch the device with their tongue when they feel the need to cough.

9. A device for the treatment of cough, the device comprising a mucoadhesive polymer, wherein the device is attached to the interior of the oral cavity of a person in need of cough treatment; and wherein a person is instructed to stimulate the device as necessary.

10. The device according to claim 9 wherein the device has a textured surface which feels rough to the tongue.
